(19) United States
(12) Patent Application Publication
(10) Pub. No.: US 2024/0273413 A1
(43) Pub. Date: Aug. 15, 2024

(54) METHOD FOR VECTORIZING MEDICAL DATA FOR MACHINE LEARNING, DATA TRANSFORMING APPARATUS AND DATA TRANSFORMING PROGRAM IMPLEMENTING THE METHOD

(71) Applicant: KaKao Healthcare Corp., Seongnam-si (KR)

(72) Inventor: Cinyoung HUR, Seongnam-si (KR)

(21) Appl. No.: 18/566,721

(22) PCT Filed: May 11, 2022
(86) PCT No.: PCT/KR2022/006758

§ 371 Date: Dec. 4, 2023

(30) Foreign Application Priority Data
Jun. 7, 2021 (KR) 10-2021-0073384

(51) Int. Cl. G06N 20/00; G16H 10/60; G16H 50/70

(52) U.S. Cl. CPC G06N 20/00; G16H 10/60; G16H 50/70

(57) ABSTRACT

Disclosed is a method of operating a data transforming apparatus, the method including: receiving patient-specific medical data and storing feature information including values of features included in the medical data into a feature data table; in the feature data table, checking at least one feature to be transformed, and looking up a feature type of each feature by referring to a feature metadata store; looking up vectorizer functions mapped to the feature type by referring a vectorizer store, and determining a set of vectorizer functions for each feature based on a designated vectorizer function decision rule and a feature attribute; generating transformed data by applying at least one specified vectorizer function to the feature to be transformed, according to a transformation condition set for each vectorizer function; and generating training data for an artificial intelligence model by using the generated transformed data.

### TECHNICAL FIELD

The present disclosure relates to data transformation for machine learning.

### BACKGROUND ART

Research is being conducted to machine learn an artificial intelligence model with medical data and to obtain various prediction results from input medical data by using the trained artificial intelligence model. However, medical data stores various attributes, such as age, gender, primary diagnosis, secondary diagnosis, diagnosis date, medication name, dosage, prescription date, imaging result, and functional test, in a table structure, and the dimension of medical data varies from patient to patient because the attributes are various depending on patient. In addition, even for the same patient, the dimensions of medical data may change over time due to increase in diagnoses or increase in drug names, the time at which data is recorded is irregular, and the pattern of medical data may change rapidly due to a pandemic.

According to the nature of medical data, it is not easy to consistently transform the medical data for both training and serving of the machine learning. While it is possible to transform large amounts of medical data accumulated up to a certain point in time into input data for an artificial intelligence model, it is difficult to transform medical data in real time after the artificial intelligence model is deployed in the same manner. On the other hand, recent studies have attempted to train artificial intelligence models by using medical data from various sites, but it is not easy to transform the medical data from various sites into standardized input data due to the different formats in which medical data is stored by different sites.

### DISCLOSURE

### Technical Problem

The present disclosure provides a method of vectorizing medical data for machine learning, and data transforming apparatus and data transforming program for implementing the same.

Specifically, the present disclosure provides a method of selecting vectorizer functions for features of input medical data and transforming the features with the selected vectorizer functions by using a feature metadata store storing features and feature types extracted from medical data, and a vectorizer store storing a vectorizer function for each feature type.

The present disclosure provides a method of vectorizing features with vectorizer functions mapped to features of input medical data, and generating input data for an artificial intelligence model by using the vectorized data.

### Technical Solution

An exemplary embodiment provides a method of operating a data transforming apparatus, the method including: receiving patient-specific medical data and storing feature information including values of features included in the medical data in a feature data table; into the feature data table, checking at least one feature to be transformed, and looking up a feature type of each feature by referring to a feature metadata store; looking up vectorizer functions mapped to the feature type by referring a vectorizer store, and determining a set of vectorizer functions for each feature based on a designated vectorizer function decision rule and a feature attribute; generating transformed data by applying at least one specified vectorizer function to the feature to be transformed, according to a transformation condition set for each vectorizer function; and generating training data for an artificial intelligence model by using the generated transformed data.

The feature metadata store may store a feature type of each feature extracted from the medical data, and the feature type may be at least one of categorical, numerical, timedelta, Boolean, and date/time.

The vectorizer store may store a plurality of vectorizer functions available for each feature type, and a transformation condition for transforming a feature for each vectorizer function.

The generating of the transformed data may include setting a real-time vectorization mode or a batch vectorization mode, and transforming the feature to be transformed with the corresponding vectorizer function according to the set mode.

The method may further include receiving feedback on prediction performance of the artificial intelligence model, and updating the vectorizer function decision rule to determine a set of vectorizer functions of features for optimizing the prediction performance.

The method may further include storing different types of artificial intelligence models generated with training data of various input data structures and generation information of each artificial intelligence model. The generation information of each artificial intelligence model may include a set of optimized features used for training and a set of vectorizer functions applied to the set of features.

The medical data may include at least one of demographic data, diagnosis data, visit history data, visit info data, lab test data, medication data, vital sign data, clinical imaging data, and functional test data.

The generating of the training data may include waiting until input data of the artificial intelligence model is completed by combining the transformed data, and using the completed input data as training data for the artificial intelligence model.

Another exemplary embodiment provides a method of operating a data transforming apparatus, the method including: receiving patient-specific medical data and storing feature information including values of features included in the medical data into a feature data table; in the feature data table, checking at least one feature to be transformed, and looking up a feature type of each feature by referring to a feature metadata store; looking up vectorizer functions mapped to the feature type by referring a vectorizer store, and determining a set of vectorizer functions for each feature based on a designated vectorizer function decision rule and a feature attribute; temporarily storing each feature in a queue, waiting until a transformation condition for a vectorizer function of the corresponding feature is satisfied, and when the transformation condition is satisfied, applying the vectorizer function to the feature stored in the queue to generate transformed data; and storing the transformed data accumulated over time, and when input data of an artificial intelligence model is completed by combining the transformed data, inputting the completed input data into the artificial intelligence model.

The feature metadata store may store a feature type of each feature extracted from the medical data, and the feature type may be at least one of categorical, numerical, timedelta, Boolean, and date/time.

The vectorizer store may store a plurality of vectorizer functions available for each feature type, and a transformation condition for transforming a feature for each vectorizer function.

The vectorizer function decision rule may be set to determine a set of vectorizer functions for each feature that optimizes performance of the artificial intelligence model.

Still another exemplary embodiment provides a computer program stored in a computer-readable storage medium and comprising instructions for causing at least one processor to execute: receiving patient-specific medical data and storing feature information including values of features included in the medical data in a feature data table; in the feature data table, checking at least one feature to be transformed, and looking up a feature type of each feature by referring to a feature metadata store; looking up vectorizer functions mapped to the feature type by referring a vectorizer store, and determining a set of vectorizer functions for each feature based on a designated vectorizer function decision rule and a feature attribute; generating transformed data by applying at least one specified vectorizer function to the feature to be transformed, according to a transformation condition set for each vectorizer function; and generating input data for an artificial intelligence model by using the generated transformed data.

The feature metadata store may store a feature type of each feature as at least one of categorical, numerical, timedelta, Boolean, and date/time. The vectorizer store may store a plurality of vectorizer functions available for each feature type, and a transformation condition for transforming a feature for each vectorizer function.

The computer program may further include instructions for causing the at least one processor to execute: receiving feedback on prediction performance of the artificial intelligence model trained by using the input data, and updating the vectorizer function decision rule to determine a set of vectorizer functions of features for optimizing the prediction performance; and storing different types of artificial intelligence models generated with input data of various structures and generation information of each artificial intelligence model.

The generating of the transformed data may include, for a real-time vectorization mode, temporarily storing each feature in a queue, waiting until a transformation condition set in a vectorizer function of the corresponding feature is satisfied, and when the transformation condition is satisfied, applying the vectorizer function to the feature stored in the queue to generate transformed data.

The generating of the input data may include waiting until the input data is completed by combining the transformed data, and inputting the completed input data into the artificial intelligence model.

### Advantageous Effects

According to the exemplary embodiments, it is possible to automate data generation pipelines for artificial intelligence models by using the feature metadata store and the vectorizer store that stores vectorizer functions for each feature type.

According to the exemplary embodiments, it is possible to preprocess medical data in a standardized manner by centrally defining features and vectorizer functions required for training and application of artificial intelligence models in the feature metadata store and the vectorizer store, and transforming medical data by referring to the feature metadata store and the vectorizer store.

According to the exemplary embodiments, by setting various vectorizer functions suitable for types of features, features may be automatically transformed through the various vectorizer functions, and the optimal set of vectorizer functions may be determined based on the performance of the artificial intelligence model. Therefore, when a user arbitrarily sets a training data structure of an artificial intelligence model, the relationship between the numerous features included in the medical data is often expressed in a limited way, but according to the exemplary embodiments, it is possible to generate training data in which the relationship between the numerous features included in the medical data is expressed through various vectorizer functions.

According to the exemplary embodiments, by transforming medical data by referring to the feature metadata store and the vectorizer store, it is possible to generate the same input data for a training phase and an application phase of an artificial intelligence model.

### DESCRIPTION OF THE DRAWINGS

FIG. 1 is a diagram illustrating a data transforming apparatus.

Each of FIGS. 2 to 5 is a diagram illustrating an example of data transformation.

FIG. 6 is a diagram illustrating an example of real-time data transformation.

FIG. 7 is a diagram illustrating data transformation for a distributed artificial intelligence model.

FIG. 8 is a flowchart of a data transforming method for training an artificial intelligence model.

FIG. 9 is a flowchart of a real-time data transforming method.

FIG. 10 is a hardware diagram of a computing apparatus according to an exemplary embodiment.

### MODE FOR INVENTION

Hereinafter, exemplary embodiments of the present invention will be described with reference to accompanying drawings so as to be easily understood by a person ordinary skilled in the art. The present disclosure can be variously implemented and is not limited to the following exemplary embodiments. Accordingly, the drawings and description are to be regarded as illustrative in nature and not restrictive. Like reference numerals designate like elements throughout the specification.

Throughout the specification, unless explicitly described to the contrary, the word “comprise”, and variations such as “comprises” or “comprising”, will be understood to imply the inclusion of stated elements but not the exclusion of any other elements. In addition, the terms “-er”, “-or”, and “module” described in the specification mean units for processing at least one function and operation, and can be implemented by hardware components or software components, and combinations thereof.

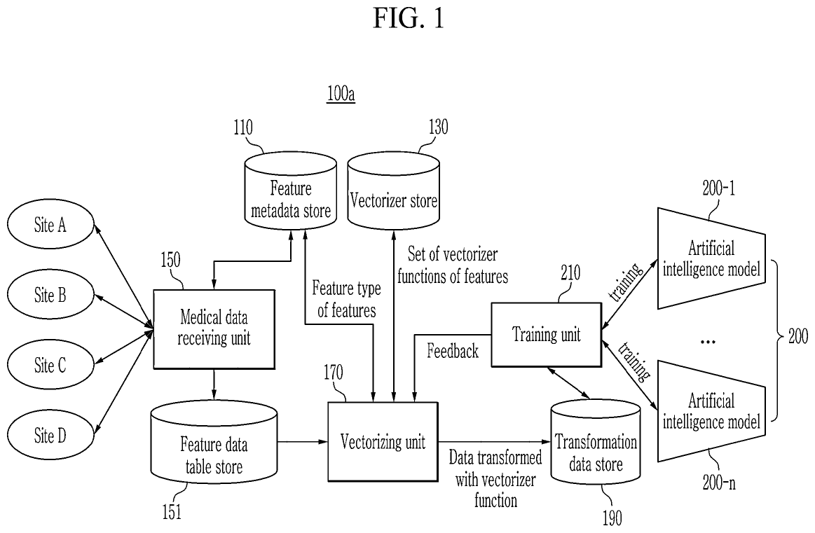

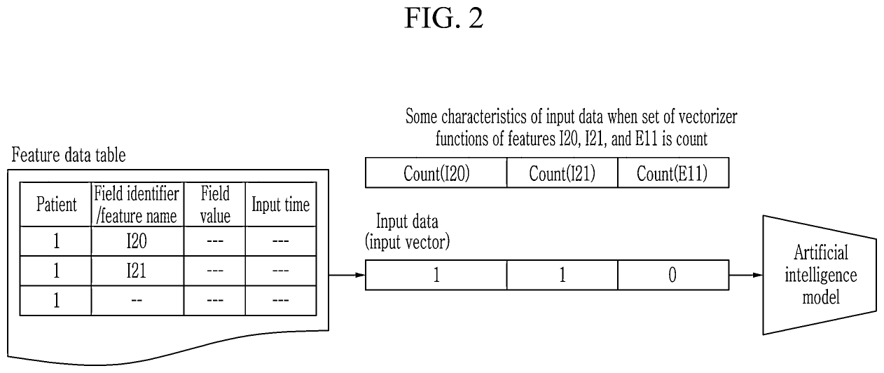

FIG. 1 is a diagram illustrating a data transforming apparatus.

Referring to FIG. 1, a data transforming apparatus 100a, which is operated by at least one processor, preprocesses medical data to generate training data for training of an artificial intelligence model 200. To this end, the data transforming apparatus 100a may include a feature metadata store 110 for storing features and feature types extracted from medical data, and a vectorizer store 130 for storing a vectorizer function for each feature type, a medical data receiving unit 150, and a vectorizing unit 170.

The feature data table generated by the medical data receiving unit 150 may be stored in the feature data table store 151. The transformed data generated by the vectorizing unit 170 may be stored in a transformation data store 190. The transformed data stored in the transformation data store 190 may be used as training data for training the artificial intelligence model 200. In the present disclosure, features may be organized hierarchically, and a set of child features (for example, emergency visits, hospitalization visits, and outpatient visits) may be a parent feature (for example, visit).

A training unit 210 trains the artificial intelligence model 200 by using the transformed data stored in the transformation data store 190. Here, depending on the features transformed in the vectorizing unit 170 and the set of vectorizer functions applied to the features, the generated artificial intelligence model 200 may vary. On the other hand, the data transforming apparatus 100a may be implemented including the training unit 210, and may not include the training unit 210 as necessary.

The feature metadata store 110 stores feature types for each feature extracted from the medical data. Features are extracted from various types of medical data, which may include, for example, demographic data, diagnosis data, visit history data, visit info data, lab test data, medication data, vital sign data, clinical imaging data, and functional test data. Imaging data may include disease-specific images (for example, coronary angiograms), reading results thereof, and the like. Functional test data may include, for example, exercise load tests.

The feature metadata store 110 stores metadata of features extracted from the medical data. Metadata may store field identifiers, feature names (field names), and feature types assigned to features in the medical data, as shown in Table 1. Feature types may be categorized as categorical, numerical, timedelta, Boolean, date/time, or any combination thereof.

TABLE 1

| | Field | | |
|---|---|---|---|
| Data type | identifier | Feature name (field name) | Feature type |
| Demographic data | 1111 | Gender | Categorical |
| Demographic data | 1112 | Blood type | Categorical |
| Demographic data | 1113 | Residence | Categorical |
| . . . | . . . | . . . | . . . |
| Diagnosis data | 2221 | Diagnosis code 120 | Categorical |
| Diagnosis data | 2233 | Diagnosis code N18 | Categorical |
| . . . | . . . | . . . | . . . |
| Visit history data | 3111 | Emergency visit | Categorical |
| Visit history data | 31234 | Outpatient visit | Categorical |
| . . . | . . . | . . . | . . . |
| Visit info data | 4367 | Medical specialty CV | Categorical |
| Visit info data | 4456 | Medical specialty NPH | Categorical |
| . . . | . . . | . . . | . . . |
| Lab test data | 5156 | Total protein | Numerical |
| Lab test data | 5233 | Troponin I | Numerical |
| . . . | . . . | . . . | . . . |
| Drug data | 6111 | Aspirin | Numerical |
| . . . | . . . | . . . | . . . |
| Vital sign data | 7111 | Systolic Blood Pressure | Numerical |
| Vital sign data | 7112 | Diastolic Blood Pressure | Numerical |
| Vital sign data | 7234 | Pulse | Numerical |
| . . . | . . . | . . . | . . . |

The vectorizer store 130 may store a plurality of available vectorizer functions for each feature type, and may store transformation triggers for transforming features for each vectorizer function. Various vectorizer functions stored in the vectorizer store 130 may optionally be used to vectorize features. Various vectorizer functions related to one-hot-encoding, data augmentation, interpolation, embedding, and the like are stored in the vectorizer store 130.

Referring to Table 2, the vectorizer functions applicable to the numerical type may include a count function, a mean function, a sum function, a min function, a max function, and the like. Vectorizer functions applicable to the categorical type may include one-hot-encoders to transform a value of a feature to binary, a Boolean function to indicate whether a condition is satisfied, a count function, and a compressor function to transform a value of a feature in data to a lower dimension. Functions applicable to the time-differentiated type may include functions that calculate the time (month, year) from the date of birth to the present and the like. Various other vectorizer functions may be defined. For example, a time conditioned function (for example, the 60_d function, 90_d function, and 365_d function in Table 2) may be defined over which the vectorizer function is applied, and a time window of last 1 week, last 2 weeks, and last 1 month may be defined. For reference, a one-hot encoder function is a 1×N matrix (vector) used to distinguish a particular feature value from all other feature values, where the vector may be represented as 0 in all digits except for a single 1 in the digit uniquely used to identify the feature value.

TABLE 2

| | | Transform | |
|---|---|---|---|
| | | condition | |
| Feature type | Vectorizer function | (trigger) | Explanation |
| Numerical | count | >1 | Calculate the number of times a |
| | | | feature is written |
| | mean | >2 | Calculate the average of feature values |
| | sum | >2 | Calculate the sum of feature values |
| | min | >1 | Calculate the minimum value of |
| | | | features |
| | max | >1 | Calculate the maximum value of |
| | | | features |
| Categorical | one-hot-encoder | exists | Transform feature value to one-hot |
| | | | vector (Example, gender male = 10, |
| | | | gender female = 01) |
| | 60_d | exists | Existence of features within 60 days |
| | 90_d | exists | Existence of features within 90 days |
| | 365_d | exists | Existence of features within 365 days |
| | count | >1 | Calculate the number of times a |
| | | | feature is written |
| | compressor | exists | Transform feature value to lower |
| | | | dimension |
| Timedelta | month | exists | Calculate the number of months of age |
| | year | exists | Calculate the number of years of age |
| | LENGTH_OF_STAY | exists | Calculate stay time associated with |
| | | | feature |

The medical data receiving unit 150 receives input of patient-specific medical data from various apparatus including the Clinical Data Warehouse (CDW), checks the features included in the medical data, and stores feature values and input time in the feature data table. The medical data receiving unit 150 may receive input of a large amount of patient-specific medical data stored in the CDW or the like. Alternatively, when a drug is administered to a patient or a new diagnosis is made, the medical data receiving unit 150 may receive medical data including the record of the administration of 10 the drug or the new diagnosis from time to time.

Referring to Table 3, each row in the feature data table lists a field identifier (or feature name) representing the feature extracted from the medical data, the value of the feature, and the time the value was input. For example, when field identifier 5156 in the lab test data has a value of a feature (total protein) written on 2015-03-30 09:25:00 and further written on 2015-03-31 09:30:00, the medical data receiving unit 150 may generate a feature data table as shown in Table 3. When field identifier 2255 of the lab test data has a value of a feature “essential hypertension” written on 2015-03-31 11:40:00, the medical data receiving unit 150 may generate a feature data table as shown in Table 3.

TABLE 3

| | | Field identifier | | |
|---|---|---|---|---|
| Row | Patient | (corresponding | Field value/ | |
| identifier | identififer | to feature name) | Feature value | Input time |
| 1 | 1 | 5156 | 6.0 g/dL | 2015-03-30 |
| | | | | 09:25:00 |
| 2 | 1 | 5156 | 4.8 g/dL | 2015-03-31 |
| | | | | 09:30:00 |
| 3 | 1 | 2255 | Essential | 2015-03-31 |
| | | | hypertension | 11:40:00 |
| . . . | . . . | . . . | . . . | . . . |

The vectorizing unit 170 generates training data for the artificial intelligence model or input data to be input to the trained artificial intelligence model by using the feature data table stored in the medical data receiving unit 150. In the following, a method of generating training data for the artificial intelligence model will be primarily described.

The vectorizing unit 170 determines a set of vectorizer functions to be applied to the features, according to the set vectorizer function decision rules and the feature attributes described in the feature data table. In this case, the features to be vectorized may be preset in a vectorizer function decision rule, and the vectorizer function decision rule may be updated according to the input data structure of the artificial intelligence model. On the other hand, the input data may consist of a combination of a plurality of transformed data, each of which may be represented as a value of a vectorizer function applied to at least one feature. The length of the input data may vary depending on the combination of transformed data.

The input data structure of the artificial intelligence model may vary depending on the training performance of the artificial intelligence model, and in the initial training phase, the input data is generated by applying all applicable vectorizer functions to each feature, and then the set of vectorizer functions for the features may be optimized by gradually selecting the transformed data that affect the artificial intelligence model's prediction results and the vectorizer functions that generate the transformed data. In other words, the prediction performance of the artificial intelligence model depends on the training data, and according to the complex and multifaceted natures of the medical data, it is difficult to determine which vectorization needs to be applied to ensure optimal prediction performance. Even with all possible vectorization, unnecessary input values that do not affect the prediction results may be used for training, and subjective vectorization by the user does not always guarantee optimal performance of the artificial intelligence model. To solve the issues, the vectorizing unit 170 may generate training data with a set of vectorizer functions appropriate for the feature attributes and gradually change the set of vectorizer functions applied to the feature to determine an optimal set of vectorizer functions for the artificial intelligence model. As the criteria for selecting a combination of features and vectorizer functions, feature importance and feature influence on the prediction result may be used depending on the type of model. The feature influence on the predictive result may be calculated by a method of quantifying which features had a large impact on the prediction result and which features had no impact at all, for example, the shapley value may be used.

The vectorizing unit 170 identifies the features (or field identifiers corresponding to the features) in the feature data table generated by the medical data receiving unit 150, and looks up the feature type of each feature by referring to the feature metadata store 110. The vectorizing unit 170 then looks up the vectorizer functions mapped to the feature type by referring to the vectorizer store 130. In this case, the type of features transformed by the vectorizing unit 170 may be predetermined based on the purpose of the artificial intelligence model or the input data structure. In other words, the vectorizing unit 170 does not transform all features included in the medical data, but may selectively transform features that are relevant to the training of the artificial intelligence model. In this case, the features related to the training of the artificial intelligence model may be initially set by the user. Alternatively, the vectorizing unit 170 may receive feedback on the prediction performance of the artificial intelligence model and exclude the features that do not affect the prediction performance from features of interest.

The vectorizing unit 170 may transform a feature of the medical data with the vectorizer function when a transformation condition is set in the vectorizer function and the transformation condition is satisfied.

In the meantime, among the features, demographic information about gender, blood type, region and the like are fixed values, so the vectorizer function appropriate to the demographic information may be predetermined with a one-hot-encoder. In this case, the one-hot-encoder applied to gender may transform female to 01 and male to 10, or may transform the gender to 1 bit (0, 1). Similarly, the one-hot-encoder applied to blood types may transform blood type A to 0001, blood type B to 0010, blood type O to 0100, and blood type AB to 1000.

Also, among features, the vectorizer function to distinguish between types may be predetermined by the one-hot-encoder. For example, the one-hot-encoder applied to visit types may transform an outpatient visit to 0001, an emergency visit to 0010, a hospitalization visit to 0100, and a checkup visit to 1000. The vectorizer function applied to a medical subject may be determined by the one-hot-encoder.

It is assumed that the vectorizing unit 170 generates input data for the initial training phase of the artificial intelligence model. The vectorizing unit 170 then determines a set of vectorizer functions applicable to each feature based on the feature attributes.

For example, when the features are diagnosis codes, the feature type of the diagnosis codes is categorical, so in the vectorizer store 130 in Table 2, a plurality of vectorizer functions applicable to categorical types are identified, such as one-hot-encoder, 60_d, 90_d, 365_d, count, compressor, and one-hot-encoder (binary value of the diagnosis code) to obtain a transformation value based on the attribute of the diagnosis code, 60_d (whether the disease name of the diagnosis code was diagnosed within 60 days), 90_d (whether the disease name of the diagnosis code was diagnosed within 90 days), 365_d (whether the disease name of the diagnosis code was diagnosed within 365 days), and count (the number of times the disease name of the diagnosis code was diagnosed) may be determined as a set of vectorizer functions for each diagnosis code.

The set of vectorizer functions of the feature may vary while the artificial intelligence model is being trained, for example, some vectorizer functions (for example, 60_d, 90_d, and 365_d) may be excluded from that the set of vectorizer functions of the corresponding feature.

If the features are Systolic Blood Pressure (SBP) or Diastolic Blood Pressure (DBP), the feature types are numerical, so in the vectorizer store 130 in Table 2, vectorizer functions applicable to numerical types (for example, count, mean, sum, min, max) are identified, and at least one of mean (the mean value of the measured blood pressure), min (the minimum value of the measured blood pressure), and max (the maximum value of the measured blood pressure) of which the values are obtainable based on a property of the systolic blood pressure/diastolic blood pressure may be determined as a set of vectorizer functions of the systolic blood pressure/diastolic blood pressure.

When the features are visit types, such as outpatient visit, emergency visit, hospitalization visit, and checkup visit, the feature type of each visit type is categorical, so in the vectorizer store 130 in Table 2, the vectorizer functions applicable to categorical types (for example, one-hot-encoder, 60_d, 90_d, 365_d, count, and compressor) may be identified, and at least one of one-hot-encoder, 60_d, 90_d, 365_d, and count of which the values are obtainable based on an attribute of the visit type, may be determined as a set of vectorizer functions for each visit type. In addition, the set of vectorizer functions may include vectorizer functions that transform the presence or absence of a visit, without distinguishing between outpatient visit, emergency visit, hospitalization visit, or checkup visit.

When the features are drugs, such as aspirin, their feature type is numerical, so in the vectorizer store 130 of Table 2, vectorizer functions applicable to numerical types (for example, count, mean, sum, min, and max) may be checked, and at least one of count (the number of times of prescriptions for the drug), mean (average dose), sum (total dose), min (lowest dose), and max (highest dose) of which values are obtainable based on the attributes of the drug may be determined as a set of vectorizer functions for each drug. As described above, the vectorizing unit 170 determines a set of vectorizer functions applicable to each feature for training the artificial intelligence model, and transforms each feature to a certain length of transformed data (vector) by using the determined set of vectorizer functions. The transformed data is combined to generate training data for the artificial intelligence model, and the artificial intelligence model is trained. Then, the vectorizing unit 170 receives feedback on the prediction performance of the artificial intelligence model or the transformed data that affects the prediction performance of the artificial intelligence model, and based on the feedback, the set of vectorizer functions for each feature may be optimized by gradually selecting the vectorizer functions that affect the prediction performance of the artificial intelligence model.

For example, the vectorizing unit 170 may transform features by using the set of vectorizer functions for each feature, as shown in Table 4, and combine the transformed data to generate input data input to the artificial intelligence model. The vectorizing unit 170 may generate transformed data for each type of data.

TABLE 4

| | | Vectorizer | |
|---|---|---|---|
| Data type | Feature name | function | Explanation |
| Demographic | Gender | one-hot- | Female: 01 |
| | | encoder | Male: 10 |
| Demographic | Blood type | one-hot- | A: 0001 |
| | | encoder | B: 0010 |
| | | | O: 0100 |
| | | | AB:1000 |
| Demographic | Residence | one-hot- | |
| | | encoder | |
| Diagnosis data | Diagnosis code I20 | count | Angina pectoris |
| Diagnosis data | Diagnosis code I21 | count | Acute myocardial infarction |
| Diagnosis data | Diagnosis code I25 | count | Chronic ischemic heart disease |
| Diagnosis data | Diagnosis code N18 | count | Chronic kidney disease |
| Diagnosis data | Diagnosis code E11 | count | Insulin-independent diabetes |
| Diagnosis data | Diagnosis code E14 | count | Unspecified diabetes |
| Lab test | Troponin I | max | Maximum value of measured |
| | | | Troponin I (quantitative), |
| | | | blood |
| Lab test | Troponin I | mean | Average value of measured |
| | | | Troponin I (quantitative), |
| | | | blood |
| Lab test | CK-MB | max | Maximum value of measured |
| | | | CK-MB(quantitative), blood |
| Lab test | E-ANC | min | Minimum value of measured |
| | | | E-ANC |
| Lab test | IG % | mean | Average value of measured |
| | | | IG % |
| Lab test | EGFR | min | Minimum value of measured |
| | | | EGFR(CKD-EPI) |
| Lab test | Creatinine | min | Minimum value of measured |
| | | | Creatinine (quantitative), |
| | | | blood |
| Lab test | Thyroid stimulating | max | Maximum value of measured |
| | hormone | | TSH (quantitative), blood |
| Lab test | Total CO2 | count | Number of times of mention of |
| | | | measured CO₂ (quantitative), |
| | | | blood |
| Drug data | aspirin | sum | Sum of aspirin used |
| Drug data | clopidogrel | sum | Sum of clopidogrel used |
| Drug data | 5% dextrose | sum | Sum of saline used |
| Drug data | heparin sodium | sum | Sum of heparin used |
| Drug data | teprenone | sum | Sum of teprenone (stomach |
| | | | ulcer medication) used |
| Drug data | meropenem | sum | Sum of meropenem (antibiotic) |
| | | | used |
| Drug data | recombinant human | sum | Sum of aprotinin used |
| | erythropoietin | | |
| Drug data | diltiazem hcl | sum | Sum of diltiazem Used |
| Vital sign | SBPT | min | Minimum value of systolic |
| | | | blood pressure |
| Vital sign | SBPT | mean | Average value of systolic |
| | | | blood pressure |
| Vital sign | DBPT | mean | Average value of diastolic |
| | | | blood pressure |
| Vital sign | SBPT | max | Maximum value of systolic |
| | | | blood pressure |
| Vital sign | DBPT | min | Minimum value of diastolic |
| | | | blood pressure |
| Vital sign | DBPT | max | Maximum value of diastolic |
| | | | blood pressure |
| Vital sign | PRPT | count | Number of times of mention of |
| | | | pulse rate |
| Visit history | Emergency visit | 365_d | Emergency visit in 365 days |
| Visit history | Emergency visit | 180_d | Emergency visit in 180 days |
| Visit history | Hospitalization visit | 365_d | Hospitalization visit in 365 |
| | | | days |
| Visit history | Hospitalization visit | 180_d | Hospitalization in 180 days |
| Visit history | Outpatient visit | 365_d | Outpatient visit in 365 days |
| Visit history | Outpatient visit | 180_d | Outpatient visit in 180 days |
| Visit history | Outpatient visit | 90_d | Outpatient visit in 90 days |
| Visit history | Outpatient visit | 60_d | Outpatient visit in 60 days |
| Visit history | Checkup visit | 365_d | Checkup visit in 365 days |
| Visit history | All visits | 365_d | Visits in 365 days, including |
| | | | all visit types |
| Visit history | All visits | 180_d | Visits in 180 days, including |
| | | | all visit types |
| Visit history | All visits | 90_d | Visits in 90 days, including all |
| | | | visit types |
| Visit history | All visits | 60_d | Visits in 60 days, including all |
| | | | visit types |
| Visit history | All visits | 30_d | Visits in 30 days, including all |
| | | | visit types |
| Visit | Visit type | one-hot- | Emergency visit, outpatient |
| information | | encoder | visit, hospitalization visit, |
| | | | checkup visit, and the like |
| Visit | Visited Medical | one-hot- | Cardiology visit, nephrology |
| information | speciality | encoder | visit, cardiothoracic surgery |
| | | | visit, and the like |
| Visit | Age | month | Age (number of months) |
| information | | | |
| Visit | Age | year | Age (number of years) |
| information | | | |
| Visit | LENGTH_OF_STAY | hour | Time spent in the emergency |
| information | | | room |

The vectorizing unit 170 may operate in a real-time vectorization mode with a short latency time or in a batch vectorization mode with high data throughput. The real-time vectorization mode may be used primarily in the serving phase of the artificial intelligence model, and the batch vectorization mode may be used primarily in the training phase of the artificial intelligence model.

In the real-time vectorization mode, the vectorizing unit 170 may vectorize features (or field identifiers corresponding to features) that are written to the feature data table in real time. The vectorizing unit 170 checks the feature in real time when the feature is registered in the feature data table, looks up the feature type by referring to the feature metadata store 110, and determines a set of vectorizer functions to apply to the feature. Then, the vectorizing unit 170 may transform the value of the feature, depending on whether the feature satisfies the transformation condition of each vectorizer function.

Alternatively, in the batch vectorization mode, the vectorizing unit 170 may transform many features included in the feature data table at once.

On the other hand, when the vectorizing unit 170 stores the transformed data of the features included in the feature data table in the transformation data store 190, the training unit 210 may generate input data by combining the transformed data corresponding to the input data structure of the artificial intelligence model among the transformation stored in the transformation data store 190.

The training unit 210 trains the artificial intelligence model 200 by using the transformed data stored in the transformation data store 190, and may generate different kinds of artificial intelligence models depending on the input data structure of the artificial intelligence model. The training unit 210 stores, for each artificial intelligence model, output information and prediction performance of each artificial intelligence model, the set of features configuring the training data and the set of vectorizer functions applied to the set of features, the input data structure, and the like.

On the other hand, values that need to be included in the input data may not yet be stored as transformed data. In this case, the training unit 210 may wait for the input data to be completed by combining the transformed data, and use the input data completed over time as training data for the artificial intelligence model.

In addition, the training unit 210 may feedback the prediction performance of the trained artificial intelligence model, transformed data of the input data that affects the prediction result of the artificial intelligence model, and the like to the vectorizing unit 170. The vectorizatino unit 170 may then change the features that configure the input data and the set of vectorizer functions of the features to generate new transformed data from the medical data.

Each of FIGS. 2 to 5 is a diagram illustrating an example of data transformation.

Referring to FIG. 2, when a patient comes in a hospital and is diagnosed with a disease name, a disease name/diagnosis code is written in the feature data table. In this case, when some characteristics included in the input data are the numbers of times of diagnosis (count) of I20, I21, and E11 among the diagnosis names/diagnosis codes, the vectorizing unit 170 may transform the diagnosis codes I20, I21, and E11 to [1,1,0]. The artificial intelligence model 200 may be trained on a given task (for example, predicting the probability of cardiovascular disease) by using input data including [1,1,0].

On the other hand, the number of times of diagnoses (count) may be subdivided into cumulative number of times of diagnosis, the number of times of diagnosis within a certain period of time (recent), and the like.

Referring to FIG. 3, when a patient is hospitalized and prescribed drug, medication information during the hospitalization is written in the feature data table. In this case, when some of the characteristics included in the input data are the total dose (sum) and maximum dose (max) of clopidogrel, aspirin, and statin during the hospitalization, the vectorizing unit 170 may transform the medication data into [10,20,15] for the total dose and [5,8,3] for the maximum dose. By using the input data including [10,20,15,5,8,3], the artificial intelligence model 200 may be trained on a given task (for example, the relationship between a disease and a drug).

Referring to FIG. 4, when some of the characteristics included in the input data are one-hot-encoder values of drugs, the vectorizing unit 170 may transform the medication information during the hospitalization written in the feature data table with one-hot-encoders. By using input data representing the medication information, the artificial intelligence model 200 may be trained on a given task (for example, the relationship between a disease and a drug). Additionally, the vectorizing unit 170 may transform the medication information to a lower dimension by using a compressor function.

Referring to FIG. 5, when a patient is hospitalized, undergoes multiple lab tests, and the LDL cholesterol level is measured, a result of the lab test during the hospitalization is written in the feature data table. In this case, when some of the characteristics included in the input data are the number of times of LDL measurement (count), the average LDL value (mean), and the maximum LDL value (max) during the hospitalization, the vectorizing unit 170 may transform the LDL cholesterol level to [3, 110, 120]. The artificial intelligence model 200 may be trained on a given task by using the input data including [3, 110, 120].

In addition, the vectorizing unit 170 may vectorize the feature to a time window, such as last 1 week, last 2 weeks, and last 1 month. For example, when a patient is hospitalized and the amount of total protein is measured periodically during the hospitalization, the vectorizing unit 170 may transform the amount of total protein for each time interval with count, mean, min, and max functions by using the data written in the feature data table, as shown in Table 5. The artificial intelligence model 200 may be trained on a given task (for example, relationship between total protein change over time and treatment progress) by using input data including [2,5.4,4.8,6.0], [2,5.4,4.8,6.0], [2,5.4,4.8,6.0], [4,5.75,4.8,6.4], and the like.

TABLE 5

| Transformation data name | count | mean | min | max |
|---|---|---|---|---|
| Total protein over last 1 week | 2 | 5.4 | 4.8 | 6.0 |
| Total protein over last 2 weeks | 2 | 5.4 | 4.8 | 6.0 |
| Total protein over last 1 month | 2 | 5.4 | 4.8 | 6.0 |
| Total protein over last 2 months | 4 | 5.75 | 4.8 | 6.4 |
| Total protein over last 3 months | 4 | 5.75 | 4.8 | 6.4 |
| Total protein over last 6 months | 7 | 6.07 | 4.8 | 7 |

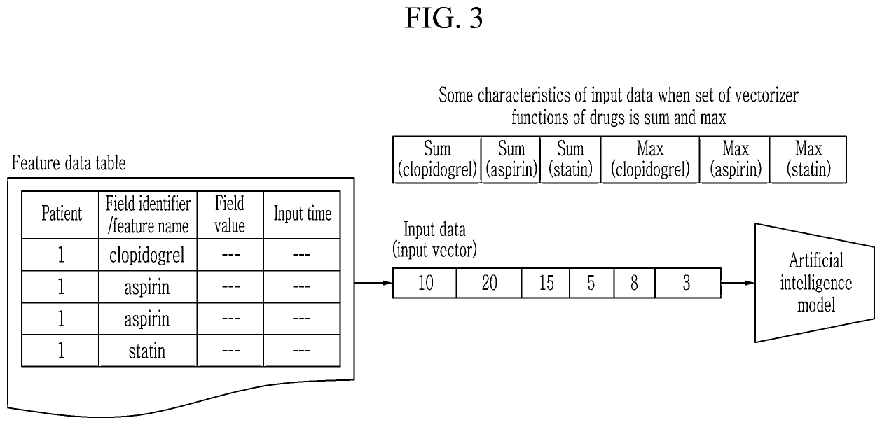

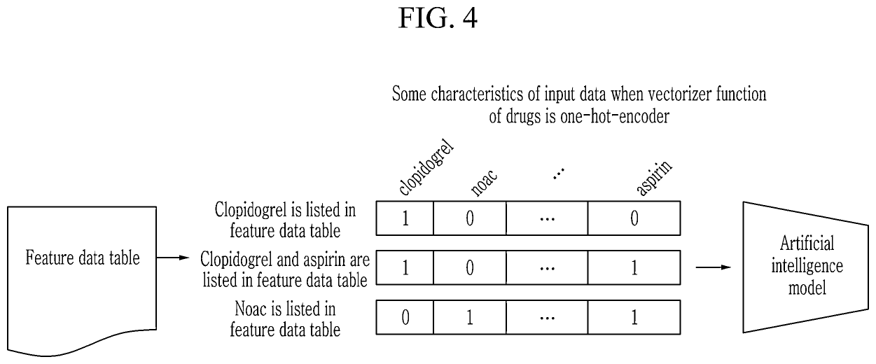

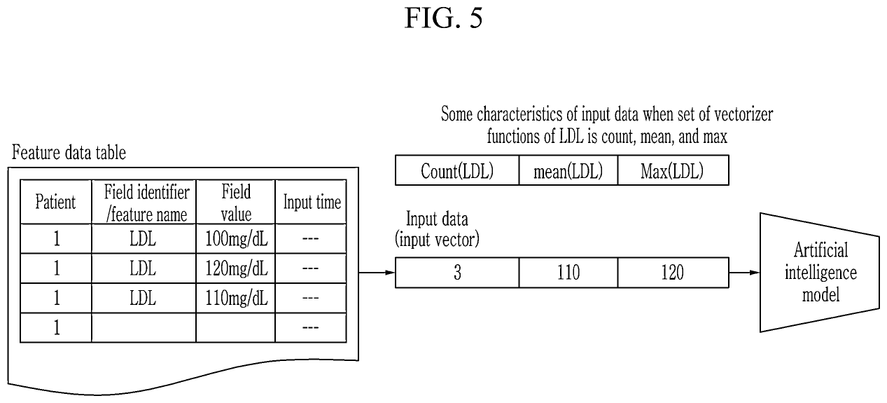

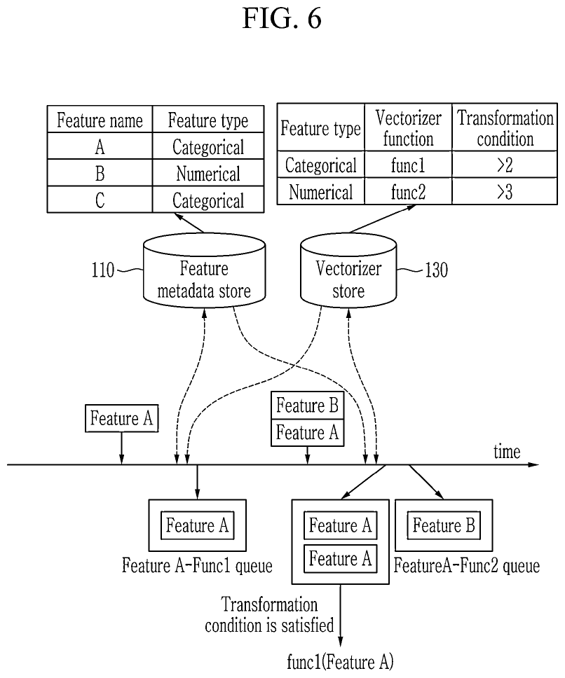

FIG. 6 is a diagram illustrating an example of real-time data transformation.

Referring to FIG. 6, the vectorizing unit 170 checks feature A written in the feature data table in real time, checks the feature type, categorical, by referring to the feature metadata store 110, and checks the vectorizer function func1 and the transformation condition (transform when there are two or more features) corresponding to the categorical feature type in the vectorizer store 130. The vectorizing unit 170 temporarily stores feature A in a featureA-func1 queue. In this case, the transformation condition of func1 is not satisfied, so the vectorizing unit 170 does not transform feature A in the featureA-func1 queue and waits for feature A to be entered.

Later, when the patient's medical data is updated, feature A and feature B may be added to the feature data table. Then, the vectorizing unit 170 temporarily stores feature A in the featureA-func1 queue, and in this case, the transformation condition of the featureA-func1 queue is satisfied, so the vectorizing unit 170 applies func1 to feature A in the featureA-func queue and transforms feature A. Depending on the transformation conditions, the vectorizing unit 170 may retrieve past feature data written in the feature data table and apply a vectorizer function.

Similarly, the vectorizing unit 170 checks feature B written in the feature data table, checks the feature type, numerical, by referring to the feature metadata store 110, and checks vectorizer function func2 and the transformation condition (transform when there are 3 or more features) corresponding to the numeric feature type in the vectorizer store 130. The vectorizing unit 170 stores feature B in a featureB-func2 queue. In this case, since the transformation condition of func2 is not satisfied, the vectorizing unit 170 does not transform feature B stored in the featureB-func2 queue, and when the data of feature B accumulates to the transformation condition, the vectorizing unit 170 applies func2 to feature B to transform feature B.

In the batch vectorization mode, the vectorizing unit 170 may check the feature As included in the feature data table, determine whether the transformation condition is satisfied, and generate transformed data of feature A.

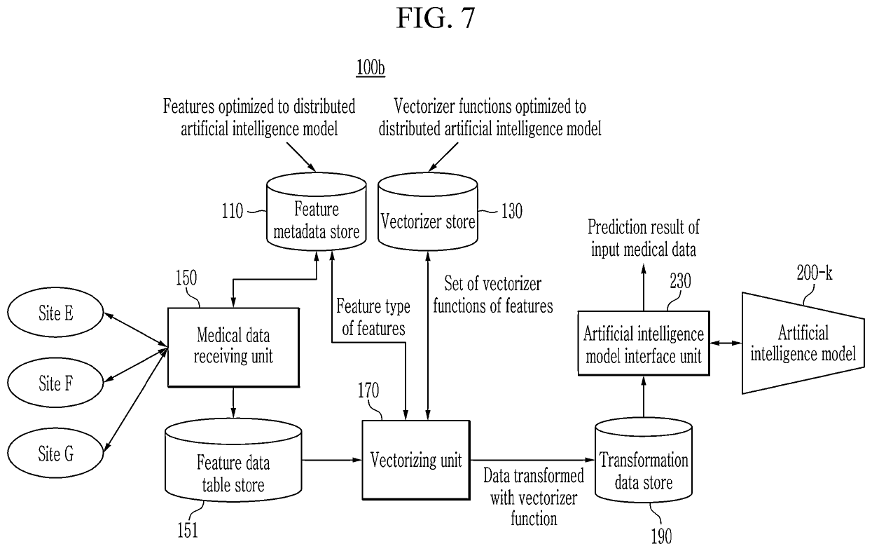

FIG. 7 is a diagram illustrating data transformation for a distributed artificial intelligence model.

Referring to FIG. 7, a data transforming apparatus 100b may be installed in a hospital, research center, or the like that wishes to obtain prediction results of medical data by using a trained artificial intelligence model 200-k. The data transforming apparatus 100b transforms the medical data into input data for the artificial intelligence model 200-k. The artificial intelligence model mounted on the data transforming apparatus 100b may be selected from various artificial intelligence models trained on the data transforming apparatus 100a.

The data transforming apparatus 100B may include a feature metadata store 110 for preprocessing medical data, a vectorizer store 130 for storing vectorizer functions for each feature type, a medical data receiving unit 150, and a vectorizing unit 170 for generating input data in a manner to generate training data for the artificial intelligence model 200-K. In this case, the information stored in the feature metadata store 110 and the vectorizer store 130 may include feature metadata and vectorizer functions optimized for the trained artificial intelligence model 200-k. The feature data table generated by the medical data receiving unit 150 may be stored in a feature data table store 151. The data generated by the vectorizing unit 170 may be stored in the transformation data store 190. Although it has been described that the data transforming apparatus 100b includes an artificial intelligence model interface unit 230 and the artificial intelligence model 200-K, the artificial intelligence model interface unit 230 and the artificial intelligence model 200-K may be implemented to be linked with the data transforming apparatus 100b.

The vectorizing unit 170 checks the features of the medical data in the feature data table generated by the medical data receiving unit 150, and looks up a feature type of each feature by referring to the feature metadata store 110. The vectorizing unit 170 then looks up the vectorizer functions mapped to the feature type by referring to the vectorizer store 130. In this case, the types of features that the vectorizing unit 170 transforms may be predetermined based on the input data structure of the trained artificial intelligence model 200-K.

When a transformation condition is set for the vectorizer function, and the transformation condition is satisfied, the vectorizing unit 170 may transform the feature of the medical data with a vectorizer function. According to the real-time data transformation method described with reference to FIG. 6, the vectorizing unit 170 checks the features written in the feature data table in real time, looks up the feature type by referring to the feature metadata store 110, and checks the vectorizer function and transformation condition corresponding to the feature type in the vectorizer store 130. The vectorizing unit 170 puts the feature into a queue in which a vectorizer function and transformation condition are set, and when the transformation condition is satisfied, the vectorizing unit 170 may transform the feature with the vectorizer function and store the transformed feature in the transformation data store 190.

Then, the artificial intelligence model interface unit 230 inputs the data stored in the transformation data store 190 into the trained artificial intelligence model 200-K and outputs the prediction result of the artificial intelligence model 200-K.

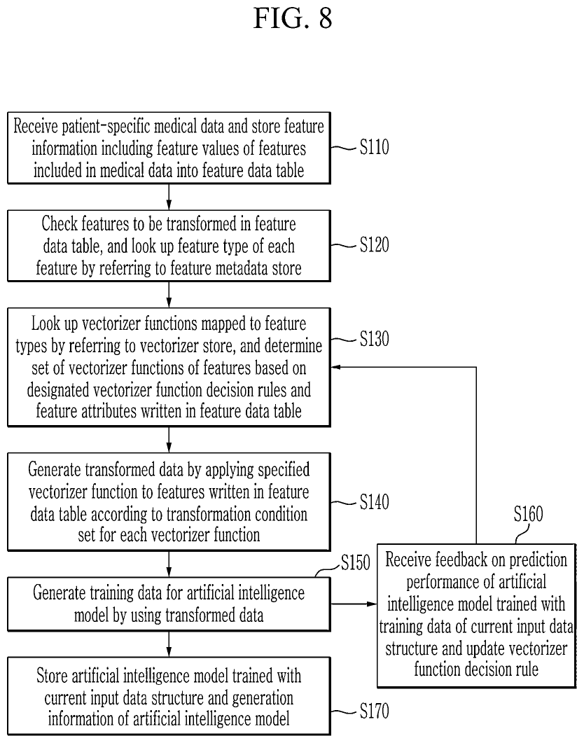

FIG. 8 is a flowchart of a data transforming method for training an artificial intelligence model.

Referring to FIG. 8, the data transforming apparatus 100a receives patient-specific medical data and stores feature information including feature values of features included in the medical data into a feature data table (S110). The data transforming apparatus 100a may receive a large amount of patient-specific medical data, or may receive updated medical data from time to time. The features in the medical data may correspond to field identifiers of the medical data. The feature data table may consist of feature names, feature values, and input times extracted from the patient-specific medical data, as shown in Table 3.

In the feature data table, the data transforming apparatus 100a checks the features to be transformed, and looks up a feature type of each feature by referring to the feature metadata store 110 (S120). The feature metadata store 110 stores metadata of the features extracted from the medical data. The feature metadata store 110 may store the field identifier, the feature name (field name), and the feature type assigned to the feature, as shown in Table 1. The feature types may be categorical, numerical, timedelta, Boolean, and date/time.

The data transforming apparatus 100a looks up the vectorizer functions mapped to the feature types by referring to the vectorizer store 130, and determines a set of vectorizer functions of the features based on the designated vectorizer function decision rules and the feature attributes written in the feature data table (S130). The vectorizer store 130 may store a plurality of available vectorizer functions for each feature type, as shown in Table 2, and may store a transformation condition for transforming the feature for each vectorizer function.

The data transforming apparatus 100a generates transformed data by applying the specified vectorizer function to the features written in the feature data table according to the transformation condition set for each vectorizer function (S140). The data transforming apparatus 100a may operate in a real-time vectorization mode with a short latency time or in a batch vectorization mode with high data throughput.

The data transforming apparatus 100a generates training data for the artificial intelligence model by using the transformed data (S150). The transformed data may be combined to match to the input data structure of the artificial intelligence model.

Thereafter, the data transforming apparatus 100a receives feedback on prediction performance of the artificial intelligence model trained with the training data of the current input data structure, and updates the vectorizer function decision rule so that a set of vectorizer functions of the features for optimizing the prediction performance is determined (S160).

Meanwhile, the data transforming apparatus 100a stores the artificial intelligence model trained with the current input data structure and generation information of the artificial intelligence model (S170). Then, the data transforming apparatus 100a may store various kinds of artificial intelligence models generated with training data of various input data structures, and the generation information of each artificial intelligence model. The generation information of each artificial intelligence model may include output information, prediction performance, an optimized set of features used in the training data and the set of vectorizer functions applied to the set of features, the input data structure, and the like.

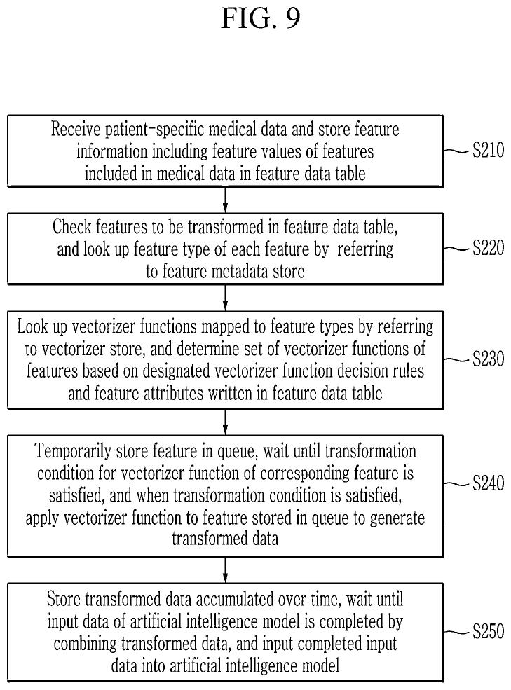

FIG. 9 is a flowchart of a real-time data transforming method.

Referring to FIG. 9, the data transforming apparatus 100b receives patient-specific medical data and stores feature information including feature values of features included in the medical data in a feature data table (S210). The data transforming apparatus 100b may receive medical data as input from time to time. The features in the medical data may correspond to field identifiers of the medical data. The feature data table may consist of feature names, feature values, and input times extracted from patient-specific medical data, as shown in Table 3.

In the feature data table, the data transforming apparatus 100b checks the features to be transformed, and looks up a feature type of each feature by referring to the feature metadata store 110 (S220). The feature metadata store 110 stores metadata of features extracted from the medical data. The feature metadata store 110 may store the field identifier, the feature name (field name), and the feature type assigned to the feature, as shown in Table 1. The feature types may be categorical, numerical, timedelta, Boolean, and date/time.

The data transforming apparatus 100b looks up the vectorizer functions mapped to the feature types by referring to the vectorizer store 130, and determines a set of vectorizer functions of the features based on the designated vectorizer function decision rules and the feature attributes written in the feature data table (S230). In this case, the vectorizer function decision rule may be set to determine a feature-specific set of vectorizer functions that optimize the performance of the trained artificial intelligence model. The vectorizer store 130 may store a plurality of available vectorizer functions for each feature type, as shown in Table 2, and may store a transformation condition for transforming the feature for each vectorizer function.

The data transforming apparatus 100b temporarily stores the feature in a queue, waits until the transformation condition set in the vectorizer function of the corresponding feature is satisfied, and when the transformation condition is satisfied, the data transforming apparatus 100b applies the vectorizer function to the feature stored in the queue to generate transformed data (S240).

The data transforming apparatus 100b stores the transformed data accumulated over time, waits until input data of the artificial intelligence model is completed by combining the transformed data, and inputs the completed input data into the artificial intelligence model (S250). When the artificial intelligence model is a trained artificial intelligence model, the data transforming apparatus 100b may obtain a prediction result output from the artificial intelligence model.

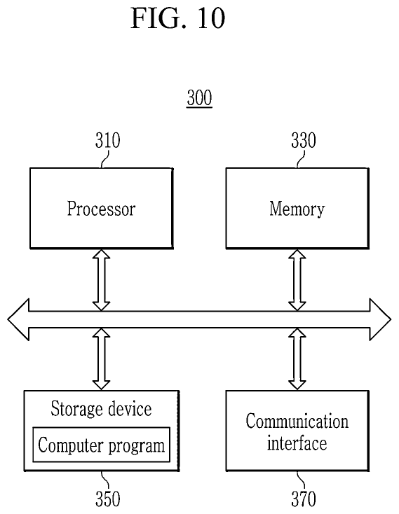

FIG. 10 is a hardware diagram of a computing apparatus according to an exemplary embodiment.

Referring to FIG. 10, the data transforming apparatus 100a and the data transforming apparatus 100b may be implemented as a computing apparatus 300 operated by at least one processor.

The computing apparatus 300 may include one or more processors 310, a memory 330 for loading computer programs executed by the processors 310, a storage apparatus 350 for storing computer programs and various data, and a communication interface 370. In addition, the computing apparatus 300 may further include various components.

The processor 310 is a apparatus that controls the operation of the computing apparatus 300 and may be various forms of processor that processes instructions contained in a computer program, and may include, for example, at least one of a Central Processing Unit (CPU), a Micro Processor Unit (MPU), a Micro Controller Unit (MCU), a Graphic Processing Unit (GPU), or any other form of processor well known in the art of the present disclosure.

The memory 330 stores various data, instructions, and/or information. The memory 330 may load a corresponding computer program from the storage apparatus 350 such that the instructions described to execute the operations of the present disclosure are processed by the processor 310. The memory 330 may be, for example, Read Only Memory (ROM) and Random Access memory (RAM).

The storage apparatus 350 may non-transitively store computer programs and various data. The storage apparatus 350 may include a non-volatile memory, such as a Read Only Memory (ROM), an Erasable Programmable ROM (EPROM), an Electrically Erasable Programmable ROM (EEPROM), and a flash memory, a hard disk, a removable disk, or any other form of computer-readable recording medium well known in the art to which the present disclosure belongs.

The communication interface 370 may be a wired/wireless communication module that supports wired/wireless communication. The communication interface 370 may access various sites that generate or store medical data.

A computer program may include instructions executed by the processor 310, and may be stored on a non-transitory computer readable storage medium, and the instructions cause the processor 310 to execute the operation of the present disclosure. The computer program may be downloaded through a network or sold as a product.

The computer program may include instructions for executing receiving patient-specific medical data and storing feature information including feature values of features included in the medical data in a feature data table, determining, from the feature data table, features to be transformed, and looking up a feature type of each feature by referring to the feature metadata store 110, looking up the vectorizer functions mapped to the feature types by referring to the vector store 130, and determining a set of vectorizer functions for the features based on the set vectorizer function decision rules and the feature attributes written in the feature data table, generating transformed data by applying the specified vectorizer functions to the features written in the feature data table according to a transformation condition set for each vectorizer function, and generating training data for an artificial intelligence model by using the transformed data.

The computer program may include instructions to further execute receiving feedback on prediction performance of the artificial intelligence model trained with the training data of a current input data structure and updating the vectorizer function decision rule to determine a set of vectorizer functions for the features to optimize the prediction performance.

The computer program may include instructions for storing various types of artificial intelligence models generated with training data of various input data structures, and instructions for storing generation information of each artificial intelligence model.

In the meantime, when the computer program is operated in a real-time vectorization mode, the computer program may include instructions for checking, from a feature data table, a feature to be transformed, and looking up a feature type of each feature by referring to the feature metadata store 110, looking up vectorizer functions mapped to the feature type by referring to the vectorizer store 130, and determining a set of vectorizer functions for the features based on a set vectorizer function decision rule and feature attributes written in the feature data table, and temporarily storing the feature in a queue, waiting until a transformation condition set for the vectorizer function of the feature is satisfied, and, when the transformation condition is satisfied, applying the vectorizer function to the feature stored in the queue to generate transformed data.

The computer program for serving a trained artificial intelligence model may include instructions for waiting for input data of the artificial intelligence model to be completed by combining the transformed data and inputting the the completed input data into the artificial intelligence model.

The exemplary embodiments of the present disclosure described above are not only implemented through the apparatus and method, but may also be implemented through programs that realize functions corresponding to the configurations of the exemplary embodiment of the present disclosure, or through recording media on which the programs are recorded.

Although an exemplary embodiment of the present invention has been described in detail, the scope of the present invention is not limited by the exemplary embodiment. Various changes and modifications using the basic concept of the present invention defined in the accompanying claims by those skilled in the art shall be construed to belong to the scope of the present invention.

1. A method of operating a data transforming apparatus, the method comprising:
receiving patient-specific medical data and storing feature information including values of features included in the medical data into a feature data table;
in the feature data table, checking at least one feature to be transformed, and looking up a feature type of each feature by referring to a feature metadata store;
looking up vectorizer functions mapped to the feature type by referring a vectorizer store, and determining a set of vectorizer functions for each feature based on a designated vectorizer function decision rule and a feature attribute;
generating transformed data by applying at least one specified vectorizer function to the feature to be transformed, according to a transformation condition set for each vectorizer function; and
generating training data for an artificial intelligence model by using the generated transformed data.

2. The method of claim 1, wherein the feature metadata store
stores a feature type of each feature extracted from the medical data, and
the feature type is at least one of categorical, numerical, timedelta, Boolean, and date/time.

3. The method of claim 1, wherein the vectorizer store
stores a plurality of vectorizer functions available for each feature type, and a transformation condition for transforming a feature for each vectorizer function.

4. The method of claim 1, wherein the generating of the transformed data includes:
setting a real-time vectorization mode or a batch vectorization mode; and
transforming the feature to be transformed with the corresponding vectorizer function according to the set mode.

5. The method of claim 1, further comprising:
receiving feedback on prediction performance of the artificial intelligence model; and
updating the vectorizer function decision rule to determine a set of vectorizer functions of features for optimizing the prediction performance.

6. The method of claim 5, further comprising:
storing different types of artificial intelligence models generated with training data of various input data structures and generation information of each artificial intelligence model,
wherein the generation information of each artificial intelligence model includes
a set of optimized features used for training and a set of vectorizer functions applied to the set of features.

7. The method of claim 1, wherein the medical data includes
at least one of demographic data, diagnosis data, visit history data, visit info data, lab test data, medication data, vital sign data, clinical imaging data, and functional test data.

8. The method of claim 1, wherein the generating of the training data includes:
waiting until input data of the artificial intelligence model is completed by combining the transformed data; and
using the completed input data as training data for the artificial intelligence model.

9. A method of operating a data transforming apparatus, the method comprising:
receiving patient-specific medical data and storing feature information including values of features included in the medical data into a feature data table;
in the feature data table, checking at least one feature to be transformed, and looking up a feature type of each feature by referring to a feature metadata store;
looking up vectorizer functions mapped to the feature type by referring a vectorizer store, and determining a set of vectorizer functions for each feature based on a designated vectorizer function decision rule and a feature attribute;
temporarily storing each feature in a queue, waiting until a transformation condition for a vectorizer function of the corresponding feature is satisfied, and when the transformation condition is satisfied, applying the vectorizer function to the feature stored in the queue to generate transformed data; and
storing the transformed data accumulated over time, and when input data of an artificial intelligence model is completed by combining the transformed data, inputting the completed input data into the artificial intelligence model.

10. The method of claim 9, wherein the feature metadata store
stores a feature type of each feature extracted from the medical data, and
the feature type is at least one of categorical, numerical, timedelta, Boolean, and date/time.

11. The method of claim 9, wherein the vectorizer store
stores a plurality of vectorizer functions available for each feature type, and a
transformation condition for transforming a feature for each vectorizer function.

12. The method of claim 9, wherein the vectorizer function decision rule is set to determine a set of vectorizer functions for each feature that optimizes performance of the artificial intelligence model.

13. A computer program stored in a computer-readable storage medium and comprising instructions for causing at least one processor to execute:
receiving patient-specific medical data and storing feature information including values of features included in the medical data in a feature data table;
in the feature data table, checking at least one feature to be transformed, and looking up a feature type of each feature by referring to a feature metadata store;
looking up vectorizer functions mapped to the feature type by referring a vectorizer store, and determining a designated of vectorizer functions for each feature based on a set vectorizer function decision rule and a feature attribute;
generating transformed data by applying at least one specified vectorizer function to the feature to be transformed, according to a transformation condition set for each vectorizer function; and
generating input data for an artificial intelligence model by using the generated transformed data.

14. The computer program of claim 13, wherein the feature metadata store
stores the feature type of each feature as at least one of categorical, numerical, timedelta, Boolean, and date/time, and
wherein the vectorizer store
stores a plurality of vectorizer functions available for each feature type, and a transformation condition for transforming a feature for each vectorizer function.

15. The computer program of claim 13, further comprising instructions for causing the at least one processor to execute:
receiving feedback on prediction performance of the artificial intelligence model trained by using the input data, and updating the vectorizer function decision rule to determine a set of vectorizer functions of features for optimizing the prediction performance; and
storing different types of artificial intelligence models generated with input data of various structures and generation information of each artificial intelligence model.

16. The computer program of claim 13, wherein the generating of the transformed data includes:
for a real-time vectorization mode, temporarily storing each feature in a queue;
waiting until a transformation condition set in a vectorizer function of the corresponding feature is satisfied; and
when the transformation condition is satisfied, applying the vectorizer function to the feature stored in the queue to generate transformed data.

17. The computer program of claim 16, wherein the generating of the input data includes:
waiting until the input data is completed by combining the transformed data; and
inputting the completed input data into the artificial intelligence model.
